(19) United States
(12) Patent Application Publication
(10) Pub. No.: US 2016/0151389 A1
(43) Pub. Date: Jun. 2, 2016

(54) USE OF ERGOSTATRIEN-3ß-OL

(71) Applicants: CHINA MEDICAL UNIVERSITY, Taichung City (TW); TAIPEI MEDICAL UNIVERSITY, Taipei City (TW)

(72) Inventors: Yueh-Hsiung KUO, Taichung City (TW); Che-Chih HSIAO, Taipei City (TW)

(21) Appl. No.: 14/657,681

(22) Filed: Mar. 13, 2015

(30) Foreign Application Priority Data
Nov. 28, 2014 (TW) 103141372

(51) Int. Cl. A61K 31/575

(52) U.S. Cl. CPC A61K 31/575

(57) ABSTRACT

A method for inhibiting the activation of mitogen-activated protein kinase (MAPK) signaling pathway in a neuroglial cell of a subject in need, comprising administering to the subject an effective amount of an active ingredient, wherein the active ingredient is selected from a group consisting of a compound of formula (I), a pharmaceutically acceptable ester of the compound of formula (I), and combinations thereof:

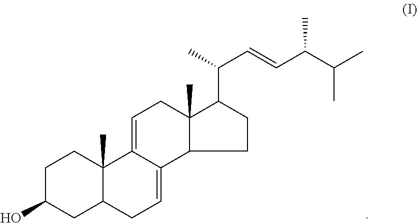 [SMILES: CC(C)[C@@H](C)/C=C/[C@@H](C)C1CCC2C3=CCC4C[C@@H](O)CC[C@]4(C)C3=CC[C@@]21C]

This application claims priority to Taiwan Patent Application No. 103141372 filed on Nov. 28, 2014, in the Taiwan Intellectual Property Office, the disclosure of which is incorporated herein in its entirety by reference.

### CROSS-REFERENCE TO RELATED APPLICATIONS

Not applicable.

### BACKGROUND OF THE INVENTION

1. Field of the Invention

The present invention relates to the use of ergostatrien-3β-ol for inhibiting the activation of mitogen-activated protein kinase (MAPK) signaling pathway in neuroglial cells of a subject in need, in particular, inhibiting the phosphorylation of c-Jun N-terminal kinase (JNK) in the neuroglial cells, inhibiting the activation of the neuroglial cells, inhibiting the expression of neuroinflammatory mediators and/or reducing the activity of matrix metalloproteinase-9 (MMP-9). The present invention especially relates to the use of ergostatrien-3β-ol to alleviate intracerebral hemorrhage (ICH)-induced injury, in particular, to alleviate cerebral nerve injury.

2. Description of the Related Art

ICH is a destructive nervous system disease with a high mortality rate and poor prognosis. The common causes of ICH are hyperlipidemia, hypertension, diabetes, hemorrhage disease, cerebral amyloid angiopathy (CAA), use of narcotics, vascular malformations, trauma, etc. ICH may break the normal blood supply for cerebrum and lead to topical defects of the nervous system, and thus, result in hemiplegia, speech disorder, coma, and even death.

Generally, when ICH is caused by the aforementioned cause(s), blood may infiltrate into the brain parenchyma and form hematomas. Then, a series of reactions, including brain edema and activation of neuroglial cells, may be induced. Various activated neuroglial cells may secrete various neuroinflammatory mediators and stimulate the activation of MMP-9, thereby, inducing a neuron-inflammatory reaction that leads to circulating inflammatory leukocyte infiltration, cerebral nerve injury, and neurological diseases.

Currently, there is no drug for effectively alleviating or preventing the ICH-induced injury. Generally, the brain hematoma is scavenged by neurosurgery to ameliorate the condition of brain edema. However, the surgery is risky, and even if patients survive the surgery, they may have severe disorders or sequelae, such as conscious coma, epilepsy, infection of the central nervous system, etc. For treating hemorrhagic stroke more effectively and safely in clinical trials, there is a need and urgency for developing a drug for alleviating ICH-induced injury. If the activation of neuroglial cells and the expression of neuroinflammatory mediators can be inhibited effectively, the ICH-induced injury can be alleviated or prevented effectively.

The inventors of the present invention found that ergostatrien-3β-ol is effective in inhibiting the activation of MAPK signaling pathway in neuroglial cells, especially in inhibiting the phosphorylation of JNK in the neuroglial cells, inhibiting the activation of the neuroglial cells, inhibiting the expressions of neuroinflammatory mediators and reducing the activity of MMP-9. Therefore, ergostatrien-3β-ol can be used to alleviate the ICH-induced injury, such as brain edema and cerebral nerve injury.

### SUMMARY OF THE INVENTION

An objective of the present invention is to provide a use of an active ingredient in the manufacture of a medicament, wherein the active ingredient is selected from a group consisting of a compound of formula (I) (i.e., ergostatrien-3β-ol), a pharmaceutically acceptable ester of the compound of formula (I), and combinations thereof:

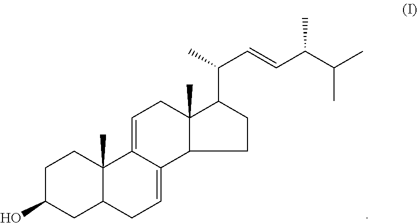 [SMILES: CC(C)[C@@H](C)/C=C/[C@@H](C)C1CCC2C3=CCC4C[C@@H](O)CC[C@]4(C)C3=CC[C@@]21C]

wherein the medicament is used for inhibiting the activation of MAPK signaling pathway in a neuroglial cell of a subject in need, especially for inhibiting the phosphorylation of JNK in the neuroglial cell.

Another objective of the present invention is to provide a method for inhibiting the activation of MAPK signaling pathway in a neuroglial cell of a subject in need, especially for at least one of inhibiting the activation of the neuroglial cell, alleviating ICH-induced cerebral nerve injury, and inhibiting and/or alleviating brain edema, comprising administering to the subject an effective amount of an active ingredient, wherein the active ingredient is selected from a group consisting of a compound of formula (I) (i.e., ergostatrien-3β-ol), a pharmaceutically acceptable ester of the compound of formula (I), and combinations thereof:

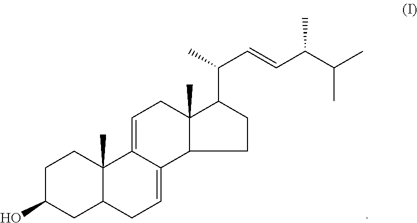 [SMILES: CC(C)[C@@H](C)/C=C/[C@@H](C)C1CCC2C3=CCC4C[C@@H](O)CC[C@]4(C)C3=CC[C@@]21C]

The detailed technology and preferred embodiments implemented for the subject invention are described in the following paragraphs for people skilled in this field to well appreciate the features of the claimed invention.

### BRIEF DESCRIPTION OF THE DRAWINGS

FIG. 1A includes western blot pictures and a statistic bar diagram, showing the effect of ergostatrien-3β-ol on inhibiting the expression level of cyclooxygenase-2 (COX-2) in microglia cells, wherein the vertical axis of the statistic bar diagram represents the relative expression level of COX-2, and the concentrations of lipopolysaccharide (LPS) and ergostatrien-3β-ol for treating different groups of cells are shown below the horizontal axis of the bar diagram;

FIG. 1B is a statistic bar diagram showing the influence of ergostatrien-3β-ol on the survival rate of microglia cells, wherein the vertical axis represents the percentage of cell survival rate, and the concentrations of ergostatrien-3β-ol for treating different groups of cells are shown below the horizontal axis of the bar diagram;

FIG. 2A includes western blot pictures and a statistic bar diagram, showing the effect of ergostatrien-3β-ol on inhibiting the activation of JNK MAPK signaling pathway in microglia cells, wherein the vertical axis of the statistic bar diagram represents the relative expression level of phosphorylated JNK1 protein, and the concentrations of LPS and ergostatrien-3β-ol for different groups of cells are shown below the horizontal axis of the bar diagram (“p-JNK” represents phosphorylated JNK1 protein; “t-JNK” represents total JNK1 protein);

FIG. 2B includes western blot pictures and a statistic bar diagram, showing the influence of ergostatrien-3β-ol on the activation of ERK MAPK signaling pathway in microglia cells, wherein the vertical axis of the statistic bar diagram represents the relative expression level of phosphorylated ERK protein, and the concentrations of LPS and ergostatrien-3β-ol for treating different groups of cells are shown below the horizontal axis of the bar diagram (“p-ERK1” represents phosphorylated ERK1 protein; “p-ERK2” represents phosphorylated ERK2 protein; “t-ERK1” represents total ERK1 protein; “t-ERK2” represents total ERK2 protein);

FIG. 3A includes a zymographic analysis picture and a statistic bar diagram, showing the effect of ergostatrien-3β-ol on reducing the activity of MMP-9 in astrocytes, wherein the vertical axis of the statistic bar diagram represents the relative activity of MMP-9, and the concentrations of LPS and ergostatrien-3β-ol for treating different groups of cells are shown below the horizontal axis of the bar diagram;

FIG. 3B includes a zymographic analysis picture and a statistic bar diagram, showing the influence of ergostatrien-3β-ol on the activity of MMP-9 in astrocytes induced by thrombin, wherein the vertical axis of the statistic bar diagram represents the relative activity of MMP-9, and the concentrations of thrombin and ergostatrien-3β-ol for treating different groups of cells are shown below the horizontal axis of the bar diagram;

FIG. 4A is a statistic bar diagram showing the brain water content of mice at 24 hours, 48 hours or 72 hours after the treatment with normal saline (NS group) or collagenase (ICH group), wherein the vertical axis represents the brain water content, [symbol] and [symbol] in each group represent the ipsilateral hemisphere and the contralateral hemisphere of the brain of the same mouse, and the ipsilateral hemisphere was treated with normal saline (NS [symbol] ) or collagenase (ICH [symbol] ) while the contralateral hemisphere group was not treated for comparison;

FIG. 4B is a statistic bar diagram showing the brain water content of mice at 48 hours after the treatment with normal saline (NS group), collagenase (ICH group) or collagenase and ergostatrien-3β-ol (“ICH+ergostatrien-3β-ol” group), wherein the vertical axis represents the brain water content, [symbol] and [symbol] in each group represent the ipsilateral hemisphere and the contralateral hemisphere of the brain of the same mouse, and the ipsilateral hemisphere was treated with normal saline (NS) [symbol] ), collagenase (ICH [symbol] ) or collagenase and ergostatrien-3β-ol (ICH+ergostatrien-3β-ol [symbol] ) while the contralateral hemisphere group was not treated for comparison;

FIG. 5 is a statistic bar diagram showing the neurobehavioral score of mice at 48 hours after the treatment with normal saline (NS group), collagenase (ICH group) or collagenase and ergostatrien-3β-ol (“ICH+ergostatrien-3β-ol” group), wherein the vertical axis represents the neurobehavioral score;

FIG. 6 includes western blot pictures and a statistic bar diagram, showing the effect of ergostatrien-3β-ol on inhibiting the expression of cyclooxygenase-2 (COX-2), wherein the vertical axis of the statistic bar diagram represents the relative expression level of COX-2; and

FIG. 7 incudes a zymographic analysis picture and a statistic bar diagram, showing the activity of MMP-9 in brain that was reduced by ergostatrien-3β-ol, wherein the vertical axis of the statistic bar diagram represents the relative activity of MMP-9.

### DETAILED DESCRIPTION OF THE INVENTION

The following will describe some embodiments of the present invention in detail. However, without departing from the spirit of the present invention, the present invention may be embodied in various embodiments and should not be limited to the embodiments described in the specification. In addition, unless otherwise stated herein, expressions “a,” “the,” and the like recited in this specification (especially in the claims) should include both the singular and plural forms. Furthermore, the term “effective amount” used in this specification refers to the amount of the compound that can at least partially alleviate the condition that is being treated in a suspected subject when administered to the subject in need. The term “subject” used in this specification refers to a mammalian, including human and non-human animals.

Unless otherwise stated herein, the term “a compound of formula (I)” used in this specification includes a compound of formula (I), a pharmaceutically acceptable ester of the compound of formula (I), and combinations thereof.

The inventors of the present invention found that the compound of formula (I) (i.e., ergostatrien-3β-ol) as below has an ability of inhibiting the activation of MAPK signaling pathway in neuroglial cells, especially of inhibiting the phosphorylation of JNK in neuroglial cells:

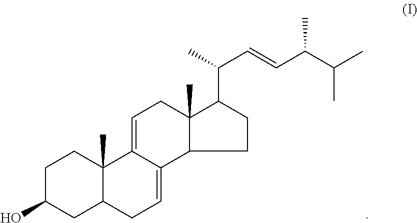 [SMILES: CC(C)[C@@H](C)/C=C/[C@@H](C)C1CCC2C3=CCC4C[C@@H](O)CC[C@]4(C)C3=CC[C@@]21C]

The inventors of the present invention also found that ergostatrien-3β-ol is effective in inhibiting brain edema.

Accordingly, the present invention relates to the use of ergostatrien-3β-ol in the manufacture of a medicament. In an embodiment of the present invention, the medicament is for inhibiting the activation of MAPK signaling pathway in neuroglial cells, and preferably for inhibiting the phosphorylation of JNK in neuroglial cells. In another embodiment of the present invention, the medicament is for inhibiting or alleviating brain edema.

Researches have proven that the activation of MAPK signaling pathway is associated with the activation of neuroglial cells, wherein c-Jun N-terminal kinase of the MAPK signaling pathway (hereinafter referred to as “JNK MAPK”) plays a critical role in the activation of the pathway. Therefore, if the MAPK signaling pathway can be inhibited, the activation of neuroglial cells, the expression of neuroinflammatory mediators and/or the activity of MMP-9 can be inhibited. Relevant description can be seen in such as “c-Jun N-terminal kinases (JNKs) mediate pro-inflammatory action of microglial. Glia. 50:235-246 (2005);” “Triptolide inhibits COX-2 expression and PGE₂ release by suppressing the activity of NF-κB and JNK in LPS-treated microglia. J. Neurochem. 107:779-788 (2008);” “Anti-neuroinflammatory activity of Kamebakaurin from Isodon japonicus via inhibition of c-Jun NH₂-terminal kinase and p38 mitogen-activated protein kinase pathway in activated microglial cells. J. Pharmacol. Sci. 116: 296-308 (2011);” and “c-Jun N-terminal kinase pathway inhibition in intracerebral hemorrhage. Cerebrovasc. 29:564-570 (2010)”, which are entirely incorporated hereinto by reference.

The medicament according to the present invention is effective in inhibiting the activation of MAPK signaling pathway in neuroglial cells, and thus, the medicament can be used to inhibit the activation of the neuroglial cells, the expression of neuroinflammatory mediators, and/or the activity of MMP-9.

Therefore, in an embodiment of the present invention, the medicament is used to inhibit the activation of neuroglial cells, such as microglial cells and astrocytes.

It has been known that the activation of neuroglial cells are associated with the pathogenesis of many neurological diseases. If the activation of neuroglial cells can be inhibited, it is favorable for the treatment or prevention of neurological diseases such as Parkinson's disease (PD), Alzheimer's disease (AD), multiple sclerosis, and cerebral ischemic. Relevant description can be seen in such as “The type 1 interleukin-1 receptor is essential for the efficient activation of microglia and the induction of multiple proinflammatory mediators in response to brain injury. J Neurosci. 22(14):6071-82 (2002)” and “US 2012/142615 A1”, which are entirely incorporated hereinto by reference. Therefore, the medicament of the present invention also can be used in the treatment or prevention of the aforementioned diseases.

In another embodiment of the present invention, the provided medicament is used to inhibit the expression of neuroinflammatory mediators and/or reduce the activity of MMP-9, wherein the neuroinflammatory mediators include, for example, cyclooxygenase-2 (COX-2).

As described herein, the occurrence of ICH-induced cerebral nerve injury was caused by the activation of neuroglial cells, the expression of neuroinflammatory mediators, and the activation of MMP-9. Therefore, if the activation of neuroglial cells and the expression of neuroinflammatory mediators can be inhibited and/or the activity of MMP-9 can be reduced, the ICH-induced cerebral nerve injury can be alleviated. In an embodiment, the medicament in accordance with the present invention is used to alleviate the ICH-induced cerebral nerve injury and/or brain edema.

The medicament in accordance with the present invention can be any suitable form and can be administered in any suitable manner. For example, the composition can be applied to a subject in need by oral administration, subcutaneous administration, nasal administration or intravenous administration, but is not limited thereby. Depending on the form and purpose, the medicament can further comprise a pharmaceutically acceptable carrier.

For instance, if the medicament in accordance with the present invention is manufactured into a dosage form suitable for oral administration, the medicament can comprise a pharmaceutically acceptable carrier that will not adversely affect the desired effect of ergostatrien-3β-ol. The carrier includes such as solvents (e.g., water, saline, dextrose, glycerol, ethanol or its analogs or a combination thereof), oily solvents, diluents, stabilizers, absorption retarders, disintegrants, emulsifiers, antioxidants, adhesives, lubricants, moisture absorbents, a solid carrier (e.g., starch and bentonite), etc. The medicament can be provided by any suitable method in any suitable oral dosage form such as a tablet, a capsule, a granule, powder, a fluid extract, a solution, a syrup, a suspension, an emulsion, a tincture, etc.

As to the dosage form suitable for subcutaneous or intravenous administration, the medicament manufactured by using ergostatrien-3β-ol in accordance with the present invention can comprise one or more components, such as an isotonic solution, a saline buffer solution (e.g., a phosphate buffer solution or a citrate buffer solution), a solubilizer, an emulsifier, 5% sugar solution, and other carriers, etc., to provide the medicament as an intravenous injection, an emulsion intravenous injection, a powder injection, a suspension injection, or a powder-suspension injection. Alternatively, the medicament manufactured by using ergostatrien-3β-ol can be prepared as a pre-injection solid. The pre-injection solid can be provided as a dosage form which is soluble in other solution(s) or suspension(s), or in an emulsifiable dosage form, and a desired injection is provided by emulsifying the pre-injection solid or dissolving it in a solution or suspension prior to being administered by the subject in need.

Optionally, the medicament manufactured by using ergostatrien-3β-ol in accordance with the present invention may further comprise one or more additives such as a flavoring agent, a toner, or a coloring agent to enhance the taste and visual perception of the medicament. A suitable amount of a preservative, a conservative, an antibacterial agent, an antifungal agent, etc., may also be added to improve the storability of the resultant medicament. In addition, the medicament may optionally further comprise one or more other active components or be used in combination with a medicament comprising the one or more active components, to further enhance the effects of the medicament or increase the application flexibility and adaptability of the formulation thus provided, as long as the other active components have no adverse effect on the desired effect of ergostatrien-3β-ol.

Depending on the requirements of the subject, the medicament manufactured by using ergostatrien-3β-ol can be applied with various administration frequencies, such as once a day, several times a day, or once for days, etc. For example, when applied to the human body for treating ICH, the dosage of the medicament is about 1 mg (as ergostatrien-3β-ol)/kg-body weight to about 50 mg (as ergostatrien-3β-ol)/kg-body weight per day, and preferably about 5 mg (as ergostatrien-3β-ol)/kg-body weight to about 30 mg (as ergostatrien-3β-ol)/kg-body weight per day, wherein the unit “mg/kg-body weight” means the dosage required per kg-body weight of the treated subject. However, for patients with acute conditions, the dosage can be increased to several times or several tens of times, depending on the practical requirements.

Accordingly, the present invention also provides a method for inhibiting the activation of MAPK signaling pathway in a neuroglial cell of a subject in need, especially for inhibiting the phosphorylation of JNK in the neuroglial cell, comprising administering to the subject an effective amount of ergostatrien-3β-ol, a pharmaceutically acceptable ester of ergostatrien-3β-ol, or combinations thereof. The method of the present invention is preferred to be used for at least one of inhibiting the activation of a neuroglial cell (such as a microglia cell, an astrocyte), inhibiting the expression of neuroinflammatory mediators (such as COX-2) and reducing the activation of MMP-9, particularly for the treatment or alleviation of the aforementioned mechanism-associated diseases. The aforementioned mechanism-associated diseases, the form and dosage for administering ergostatrien-3β-ol are all in line with the above descriptions.

In addition, the present invention provides a method for inhibiting or alleviating the brain edema, especially for inhibiting or alleviating the ICH-induced brain edema, comprising administering to a subject in need an effective amount of ergostatrien-3β-ol, a pharmaceutically acceptable ester of ergostatrien-3β-ol, or combinations thereof. The form and dosage for administering ergostatrien-3β-ol are all in line with the above descriptions.

The present invention will be further illustrated in details with specific examples as follows. However, the following examples are provided only for illustrating the present invention, and the scope of the present invention is not limited thereby.

### EXAMPLES

### A. Cellular Experiments

### Example 1

### Effect of Ergostatrien-3β-Ol on Inhibiting the Activation of Microglia Cells

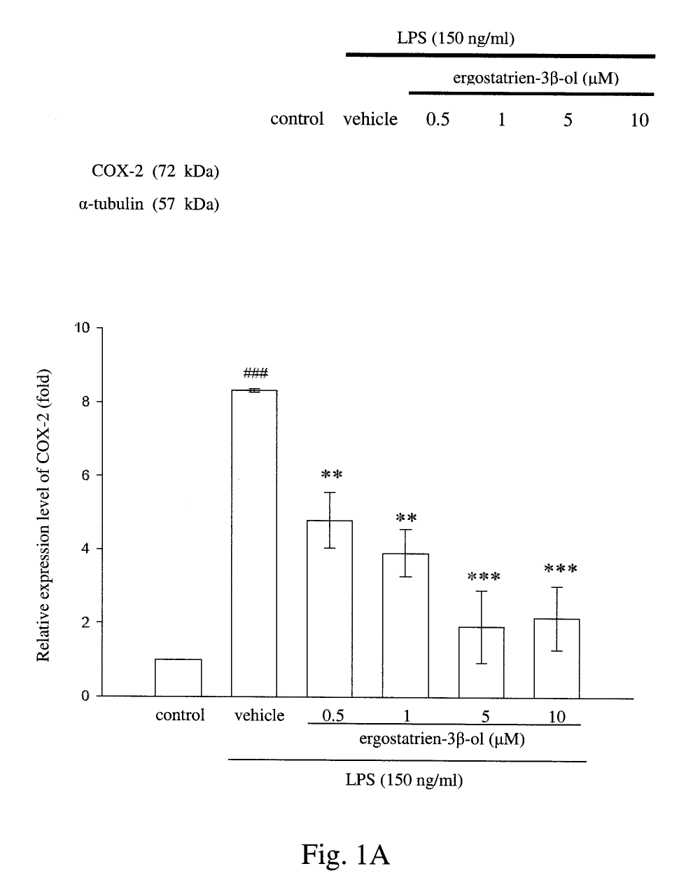

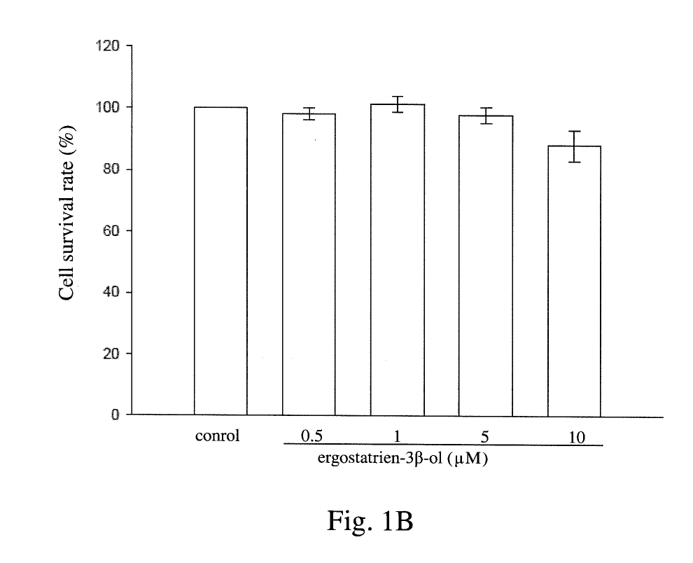

BV-2 cells (i.e., a mouse microglial cell line) were cultured in DMEM at a concentration of 2×10⁵ cells per mL of the DMEM at 37° C. with 5% CO₂ for 16 hours, wherein the DMEM was supplemented with deactivated fetal bovine serum (FBS, 10%), penicillin G (100 units/ml)/streptomycin (100 μg/ml), L-glutamine (3.65 mM), 4-(2-hydroxyethyl)-1-piperazineethanesulfonic acid (HEPES, 18 mM) and NaHCO₃ (23.57 mM). Then, the old culture medium was removed and fresh culture medium was added with a vehicle (i.e., 0.2% ethanol) or with ergostatrien-3β-ol at different concentrations (0.5, 1, 5 and 10 μM) to treat BV-2 cells for 15 minutes. Then, BV-2 cells were activated by being treated with lipopolysaccharide (LPS, 150 ng/ml) for 24 hours. BV-2 cells were lysed by a cell lysis buffer and centrifuged. The supernatant was collected and analyzed by western blot to investigate the effect of ergostatrien-3β-ol on the activation of microglia cells. The results are shown in FIGS. 1A and 1B.

As shown in FIG. 1A, the relative expression rate of COX-2 of the vehicle group (i.e., the cells treated with 0.2% ethanol, and then induced with 150 ng/ml LPS) was increased to be 8.34±0.05-fold of that of the control group (i.e., the cells treated with 0.2% ethanol, but without the induction of LPS). However, if the microglial cells were pre-treated with ergostatrien-3β-ol at a concentration of 0.5, 1, 5 or 10 μM before being induced with LPS, the relative expression rate of COX-2 in microglia cells were reduced respectively to be 4.80±0.75-fold, 3.91±0.64-fold, 1.92±0.98-fold and 2.14±0.86-fold of that of the control group. That is, the inhibition ratio were 74%, 94%, 98% and 106% respectively. The results indicates ergostatrien-3β-ol can effectively inhibit the activation of microglia cells.

In addition, as shown in FIG. 1B, both 0.2% ethanol and ergostatrien-3β-ol at a concentration of 0.5, 1, 5 or 10 μM, would not affect the survival rate of BV-2 cells. This result indicates that ergostatrien-3β-ol can inhibit the expression of neuroinflammatory mediators, but will not induce the cytotoxicity-caused side effects, and thus, can be used to provide a quality medicament for alleviating brain injury.

### Example 2

### Effect of Ergostatrien-3β-ol on Inhibiting the Activation of MAPK Signaling Pathway

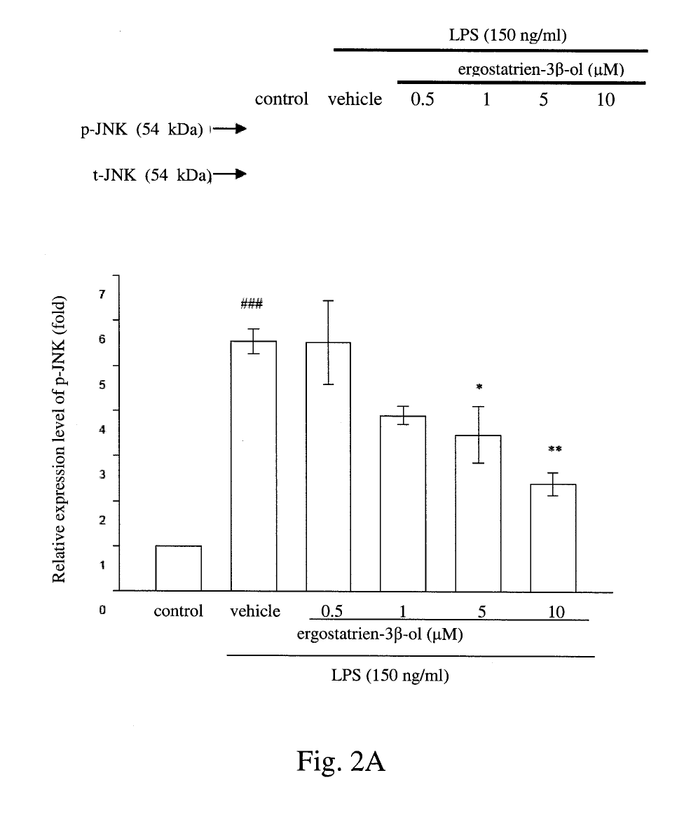

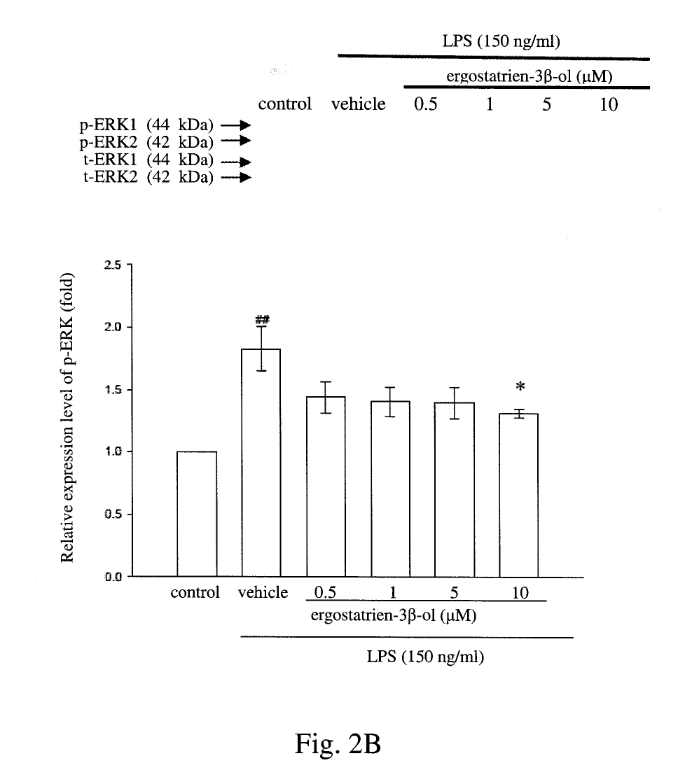

The experimental procedures in Example 1 were repeated and the effect of ergostatrien-3β-ol on the activation of MAPK signaling pathway was analyzed by western blot. The results are shown in FIGS. 2A and 2B.

As shown in FIG. 2A, the phosphorylation level of c-Jun N-terminal kinase of the MAPK signaling pathway (hereinafter referred to as “JNK MAPK”) in the vehicle group was increased to be 5.5±0.3 fold of that of the control group. However, if the microglial cells were pre-treated with ergostatrien-3β-ol at a concentration of 0.5, 1, 5 or 10 μM before being induced with LPS, the phosphorylation level of JNK MAPK in microglia cells were reduced respectively to be 5.5±0.9-fold, 3.9±0.2-fold, 3.5±0.6-fold and 2.4±0.3-fold of that of the control group. The results indicate that ergostatrien-3β-ol can effectively inhibit the activation of MAPK signaling pathway, especially the phosphorylation of JNK MAPK. On the other hand, as shown in FIG. 2B, the phosphorylation level of extracellular regulated protein kinase of the MAPK signaling pathway (hereinafter referred to as “ERK MAPK”) was inhibited by the treatment with 10 μM ergostatrien-3β-ol. The results indicate that ergostatrien-3β-ol can also inhibit the phosphorylation of ERK MAPK.

### Example 3

### Effect of Ergostatrien-3β-Ol on Inhibiting the Activation of Astrocytes

It has been known that the MMP-9-mediated gelatinolysis in astrocytes can be induced by LPS in a concentration dependent manner. The preparation and culture of astrocytes can be seen in “Preparation of separate astroglial and oligodendroglial cell cultures from rat cerebral tissue. J. Cell Biol. 85:890-902 (1980)” and “A potent antioxidant, lycopene, affords neuroprotection against microglia activation and focal cerebral ischemia in rats. In Vivo. 18:351-356 (2004)”, which are entirely incorporated hereinto by reference.

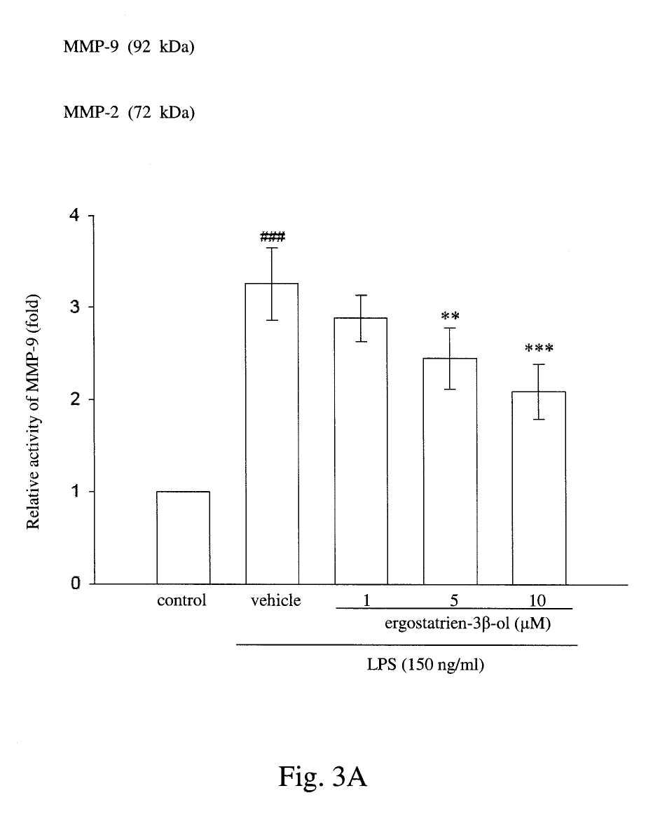

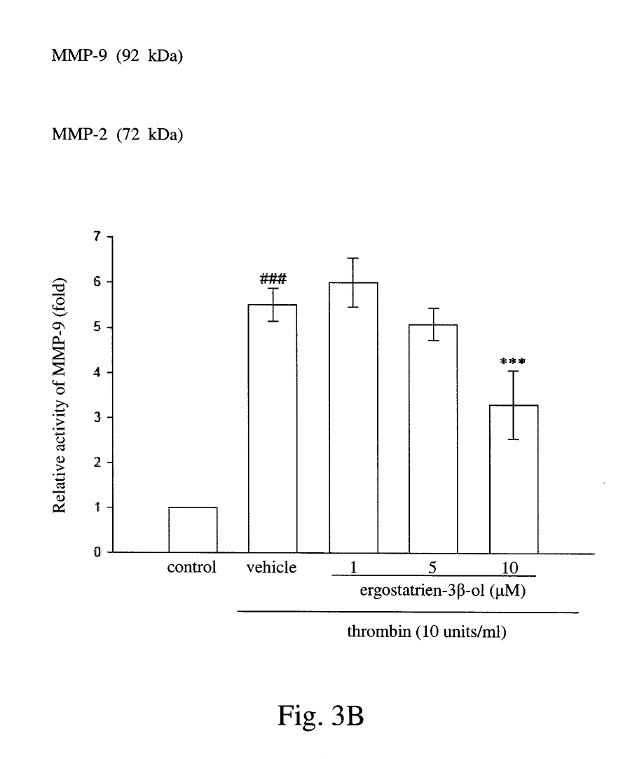

Astrocytes were respectively treated with vehicle (i.e., 0.2% ethanol) or ergostatrien-3β-ol (1, 5 and 10 μM) for 15 minutes, and then induced by LPS (500 ng/ml) or thrombin (10 units/ml) for 24 hours to induce the activation of MMP-9 in the astrocytes. Then, the MMP-9-mediated gelatinolysis was analyzed by zymographic analysis to demonstrate the effect of ergostatrien-3β-ol on the activity of MMP-9. The results are shown in FIGS. 3A and 3B.

As shown in FIG. 3A, the MMP-9-mediated gelatinolysis of the vehicle group (i.e., the cells treated with 0.2% ethanol, and then induced with 500 ng/ml LPS) was increased to be 3.3±0.4-fold of that of the control group (i.e., the cells treated with 0.2% ethanol, but without the induction of LPS). However, if the astrocytes were pre-treated with ergostatrien-3β-ol at a concentration of 0.5, 1, 5 or 10 μM before being induced with LPS, the MMP-9-mediated gelatinolysis was reduced respectively to be 2.9±0.3-fold, 2.5±0.3-fold and 2.1±0.3-fold of that of the control group. On the other hand, as shown in FIG. 3B, the MMP-9-mediated gelatinolysis of the vehicle group (i.e., the group treated with 0.2% ethanol, and then induced with 10 units/ml thrombin) was increased to be 5.5±0.4-fold of that of the control group. However, if the astrocytes were pre-treated with ergostatrien-3β-ol at a concentration of 5 or 10 μM before being induced with thrombin, the MMP-9-mediated gelatinolysis was reduced respectively to be 5.1±0.4-fold and 3.3±0.8-fold of that of the control group. The results indicate that ergostatrien-3μ-ol can effectively inhibit the activation of astrocytes.

### B. Animal Experiment

### Example 4

### ICH Induction in a Mouse Model

Male C57B2/6J mice (each weighs about 23 to 28 g) were anesthetized with chloral hydrate (400 mg/kg), and then a 1 mm burr hole was drilled 2 mm lateral to the midline and 0.3 mm anterior to the bregma of each mouse. Type VII-S collagenase (0.02 unit dissolved in 1 μl saline) was infused into the area of the caudate/putamen at a rate of 0.2 μl/min for 5 minutes. After an additional period of 5 minutes, the burr hole was occluded. The mice were allowed to recover with a controlled heating lamp and a controlled heating pad (37° C.) and an ICH mouse model was obtained, hereinafter referred to as “ICH group.” Additionally, a pseudo-animal that was handled with the same mode but only infused with an equal volume of normal saline (NS) was obtained, hereinafter referred to as “NS group.”

At 30 minutes post-collagenase injection, ergostatrien-3β-ol suspended in a 0.5% (w/v) vehicle (e.g., carboxymethyl cellulose (CMC)) was orally administered to the mouse at a dosage of 30 mg/kg. The resultant mouse was referred to as “ICH+ergostatrien-3β-ol group”. The control group was an ICH mouse that was only administered with an equal volume of vehicle, hereinafter referred to as “ICH+vehicle group.”

### Example 5

### The Effect of Ergostatrien-3β-Ol on Alleviating Brain Edema

At 24, 48 and 72 hours after the administrations of the “ICH+ergostatrien-3β-ol group” and “ICH+vehicle group” mice mentioned in Example 4, four groups of mice (i.e. the “ICH group,” “NS group,” “ICH+ergostatrien-3β-ol group” and “ICH+vehicle group”) were anesthetized with chloral hydrate (600 mg/kg) and perfused with 4° C. normal saline for 15 minutes. The brain tissues were sectioned coronally into four sequential parts from the frontal to the occipital lobe. The ipsilateral and contralateral hemispheres slices (3 mm-thick) were collected separately, wherein the ipsilateral hemisphere is the abnormal lateral (i.e., the lateral with ICH) and the contralateral hemisphere is the normal lateral (i.e., the lateral without ICH).

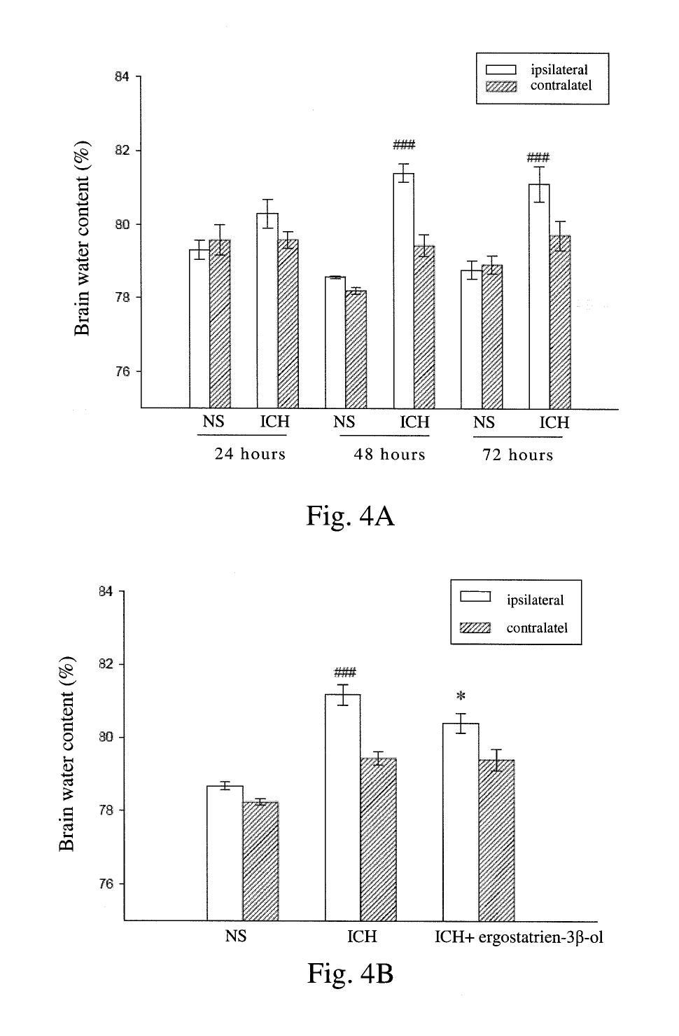

After wet weights of the brain tissues of ipsilateral hemisphere and contralateral hemisphere of the “NS group,” “ICH group” and “ICH+ergostatrien-3β-ol group”) mice were measured, the brain tissues of the ipsilateral hemisphere and contralateral hemisphere were dried at 100° C. for 24 hours to obtain the dry weights. The brain water content was calculated by the following formula: (wet weight−dry weight)/wet weight×100%. The results are shown in FIGS. 4A and 4B.

As shown in FIG. 4A, the brain water content of the ipsilateral hemisphere was significantly increased after the mouse ICH was induced by collagenase for 48 and 72 hours. As shown in FIG. 4B, at 48 hours after ICH was induced by collagenase, as compared to the brain water content of the “NS group” (78.7±0.1, p<0.001), the brain water content of the “ICH group” (81.2±0.3%, p<0.001) was significantly increased. The brain water content of the “ICH+ergostatrien-3β-ol group” (80.4±0.3%, p<0.001) was significantly less than that of the “ICH group.” The results indicate that ergostatrien-3β-ol can effectively alleviate the ICH-induced brain edema.

### Example 6

### Effect of Ergostatrien-3β-ol on Alleviating Neurobehavioral Deficits

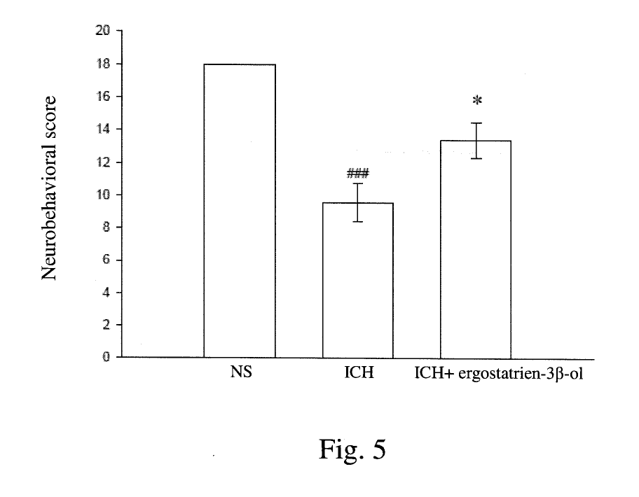

At 48 hours after ICH was induced by collagenase, three groups of mice mentioned in Example 4 (i.e., the “NS group,” “ICH group” and “ICH+ergostatrien-3β-ol group”) were analyzed by a neurobehavioral test, which includes six tests for spontaneous activity, symmetry in the movement of four limbs, forepaw outstretching, climbing, body proprioception, and response to vibrissae touch. The mean of the scores of all six individual test was referred to as a neurobehavioral score (the minimum is 3, and the maximum is 18). The lower score represents the more severe neurobehavioral deficits, while the higher score represents the alleviation of neurobehavioral deficits. The results are shown in FIG. 5.

As shown in FIG. 5, the neurobehavioral score of the “ICH group” mouse is 9.6±1.2 (p<0.05) and the neurobehavioral score of the “ICH+ergostatrien-3β-ol group” mouse is 13.4±1.1 (p<0.05). The results indicate that ergostatrien-3β-ol can alleviate the ICH-induced neurobehavioral deficits effectively.

### Example 7

### Effect of Ergostatrien-3β-Ol on Inhibiting the Expression of Neuroinflammatory Mediators

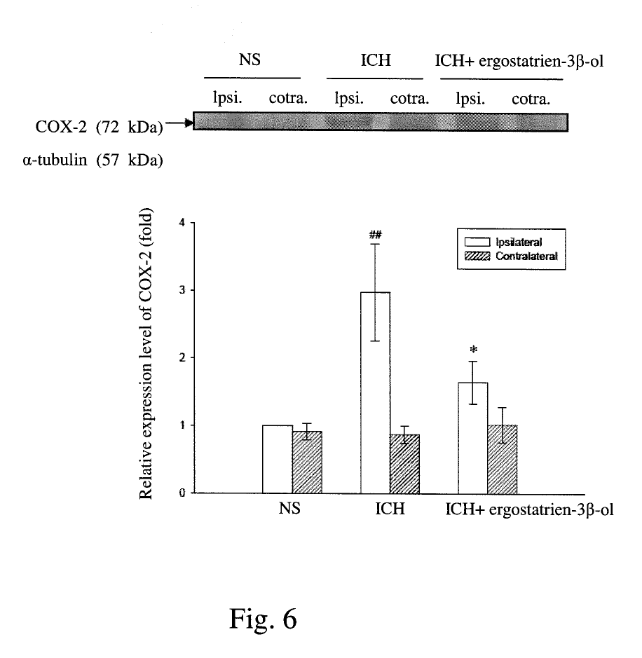

At 24 hours after ICH was induced by collagenase, the effects of ergostatrien-3β-ol on the expression of COX-2 in the brain tissues of the “NS group,” “ICH group” and “ICH+ergostatrien-3β-ol group” mice were analyzed by western blot. The results are shown in FIG. 6.

As shown in FIG. 6, as compared to the “NS group,” the expression level of COX-2 in the ipsilateral hemisphere of the “ICH group” mouse was greatly increased. However, as compared to the “ICH group,” the expression level of COX-2 in the ipsilateral hemisphere of the “ICH+ergostatrien-3β-ol group” mouse was significantly reduced. The aforementioned results indicate that ICH may stimulate the expression of neuroinflammatory mediators, while the administration of ergostatrien-3β-ol can effectively inhibit the expression of ICH-induced neuroinflammatory mediators.

### Example 8

### Effect of Ergostatrien-3β-Ol on Reducing the Ability of MMP-9

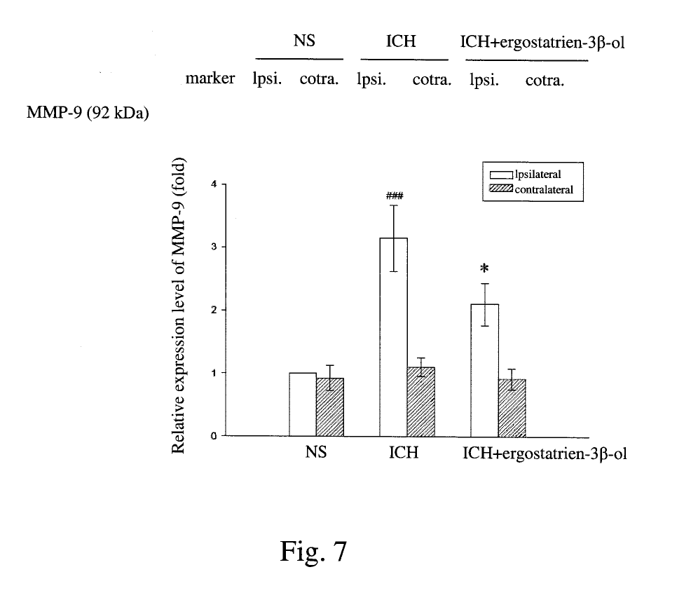

At 24 hours after ICH was induced by collagenase, the effects of ergostatrien-3β-ol on the activity of MMP-9 in the brain tissues of the “NS group,” “ICH group” and “ICH+ergostatrien-3β-ol group” mice were analyzed by zymographic analysis. The results are shown in FIG. 7. The zymographic analysis can be seen in “Dynasore, a dynamin inhibitor, induces PAI-1 expression in MeT-5A human pleural mesothelial cells. Am. J. Respir. Cell Mol. Biol. 40:629-700 (2009)”, which is entirely incorporated hereinto by reference.

As shown in FIG. 7, as compared to the “NS group,” the activity of MMP-9 in the ipsilateral hemisphere of the “ICH group” mouse was greatly increased. However, as compared to the “ICH group,” the activity of MMP-9 in the ipsilateral hemisphere of the “ICH+ergostatrien-3β-ol group” mice was significantly reduced. The aforementioned results indicate that ICH may promote the activation of MMP-9, while the administration of ergostatrien-3β-ol can effectively reduce the activity of MMP-9.

1. A method for inhibiting the activation of mitogen-activated protein kinase (MAPK) signaling pathway in a neuroglial cell of a subject in need, comprising administering to the subject an effective amount of an active ingredient, wherein the active ingredient is selected from a group consisting of a compound of formula (I), a pharmaceutically acceptable ester of the compound of formula (I), and combinations thereof:

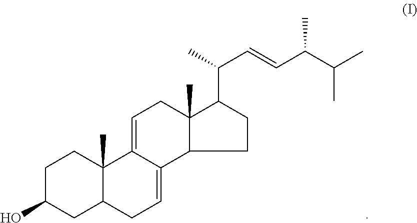 [SMILES: CC(C)[C@@H](C)/C=C/[C@@H](C)C1CCC2C3=CCC4C[C@@H](O)CC[C@]4(C)C3=CC[C@@]21C]

2. The method as claimed in claim 1, which is for inhibiting the phosphorylation of c-Jun N-terminal kinase (JNK) in the neuroglia cell.

3. The method as claimed in claim 1, which is for inhibiting the activation of the neuroglia cell.

4. The method as claimed in claim 3, wherein the neuroglia cell is at least one of a microglia cell and an astrocyte.

5. The method as claimed in claim 1, which is for inhibiting the expression of neuroinflammatory mediators and/or reducing the activity of matrix metalloproteinase-9.

6. The method as claimed in claim 5, wherein the neuroinflammatory mediator is cyclooxygenase-2 (COX-2).

7. The method as claimed in claim 1, wherein the subject is suffering from intracerebral hemorrhage (ICH).

8. The method as claimed in claim 1, which is for alleviating intracerebral hemorrhage (ICH)-induced cerebral nerve injury.

9. The method as claimed in claim 1, which is for treating or preventing neurological diseases.

10. The method as claimed in claim 1, wherein the active ingredient is administered at an amount ranges from about 1 mg (as the compound of formula (I))/kg-body weight to about 50 mg (as the compound of formula (I))/kg-body weight.

11. The method as claimed in claim 10, wherein the active ingredient is administered at an amount ranges from about 5 mg (as the compound of formula (I))/kg-body weight to about 30 mg (as the compound of formula (I))/kg-body weight.

12. A method for inhibiting or alleviating intracerebral hemorrhage (ICH)-induced brain edema, comprising administering to a subject in need an effective amount of an active ingredient, wherein the active ingredient is selected from a group consisting of a compound of formula (I), a pharmaceutically acceptable ester of the compound of formula (I), and combinations thereof:

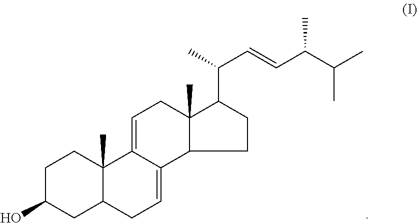 [SMILES: CC(C)[C@@H](C)/C=C/[C@@H](C)C1CCC2C3=CCC4C[C@@H](O)CC[C@]4(C)C3=CC[C@@]21C]

13. (canceled)

14. The method as claimed in claim 12, wherein the active ingredient is administered at an amount ranges from about 1 mg (as the compound of formula (I))/kg-body weight to about 50 mg (as the compound of formula (I))/kg-body weight.

15. The method as claimed in claim 14, wherein the active ingredient is administered at an amount ranges from about 5 mg (as the compound of formula (I))/kg-body weight to about 30 mg (as the compound of formula (I))/kg-body weight.
